(19) United States
(12) Patent Application Publication
(10) Pub. No.: US 2006/0229679 A1
(43) Pub. Date: Oct. 12, 2006

(54) External defibrillator and a method of determining when to use the external defibrillator

(76) Inventor: Tae Hong Joo, Redmond, WA (US)

Correspondence Address: Ms. Mary Y. Redman, Esq.;MEDTRONIC EMERGENCY RESPONSE SYSTEMS INC., 11811 Willows Road N.E., P.O. Box 97006, Redmond, WA, 98073-9706, US

(21) Appl. No.: 11/095,304

(22) Filed: Mar. 31, 2005

(51) Int. Cl. A61N 1/39

(52) U.S. Cl. 607/5

(57) ABSTRACT

In a method of operating an external defibrillator configured to provide a defibrillation shock to a patient, physiological data is gathered from the patient. Next, the physiological data is analyzed using a first algorithm to determine whether to initiate a shock. Then, if it is determined that a defibrillation shock should be provided, the physiological data is analyzed using a second algorithm to verify the determination to shock.

### TECHNICAL FIELD OF THE INVENTION

This invention relates to the field of defibrillators, and more particularly, relates to an external defibrillator and a method of determining when to use the external defibrillator to apply a shock.

### BACKGROUND OF THE INVENTION

The human heart is responsible for pumping blood throughout the body. The heart consists of four chambers; a left and right atrium located near the top of the heart, and a left and right ventricle located near the bottom of the heart. The heart is controlled by an electrical system. The healthy heart pumping pattern is known as the sinus rhythm. The sinus rhythm is controlled by electrical signals generated at the sinusoidal (SA) node, which is located in the right atrium. The electrical signals produced by the SA first causes the left and right atria to contract, pumping blood into the ventricles. The electrical signals then cause the ventricles to contract, pumping the blood to the lungs for oxygenation (for the right ventricle) and pumping oxygenated blood throughout the body (for the left ventricle). In an average day a typical person's heart beats 100,000 times, pumping about 2,000 gallons of blood.

In certain circumstances, the heart's normal electrical system can malfunction, which can result in an irregular heartbeat. An irregular heartbeat can result in improper heart function. An irregular heartbeat is generally referred to as an arrhythmia.

One type of arrhythmia is ventricular fibrillation (VF). When the heart is undergoing VF, the ventricles of the heart suddenly develop a rapid, irregular heartbeat that results in quivering ventricles that are unable to pump blood. A patient experiencing VF will experience a loss of pulse and become unconscious within a matter of seconds. Ventricular fibrillation is the most common cause of sudden cardiac arrest (SCA).

The most effective emergency treatment for VF is the delivery of an electrical shock to restart the patient's heart. The electrical shock can be delivered by a device called a defibrillator. Typically, voltage is applied to the patient through the defibrillator's electrodes or paddles, which are placed on the patient's body. The applied voltage results in an electrical current that flows through the heart. This electrical current can halt the VF, allowing normal heart rhythm to return. This process is known as defibrillation.

Survival rates from VF are higher the sooner defibrillation is performed. Different types of defibrillators exist. One type of device that has been developed to provide rapid access to defibrillation is the Automated External Defibrillator or Automatic External Defibrillator, both referred to by the acronym AED. A typical AED is a portable device that analyzes the patient's heart's rhythm and either delivers an electric shock if needed or prompts the user to deliver an electric shock if needed. The need to deliver an electrical shock can be determined by analyzing the heart's rhythm using an algorithm to determine whether to shock. Certain AED's provide audio and/or visual prompts to assist the user of the AED.

In order to reduce the time between the onset of VF and the initiation of defibrillation, AED's are being placed in a variety of public and private settings, such as shopping malls, aircrafts and the like. Some AED's have become available for purchase by individuals for home use. The widespread deployment of AED's helps to reduce the time between the onset of VF and the initiation of defibrillation.

AED's are designed to provide a shock only if the AED determines that a shock is needed. This is done by examining physiological signals of the patient that are sensed from the electrodes of the AED that are placed on the patient. In certain AED's, the electrical activity of the patient's heart is detected and converted into an electrocardiogram (ECG) waveform. The ECG waveform is then evaluated using an algorithm to determine if the application of a shock is needed. While current algorithms can accurately determine when to shock, there are cases where a shock is applied to a patient when it might have been better not to shock. The ability of a defibrillator to recognize a non-shockable event and not shock it is known as specificity. Therefore, what is needed is a method and system for increasing the specificity of defibrillators.

### SUMMARY OF THE INVENTION

In one embodiment of the present invention, a method of operating an external defibrillator configured to provide a defibrillation shock to a patient is disclosed. In the method, physiological data is gathered from the patient. Next, the physiological data is analyzed using a first algorithm to determine whether to initiate a shock. Then, if it is determined that a defibrillation shock should be provided, the physiological data is analyzed using a second algorithm to verify the determination to shock.

In another embodiment, an external defibrillator is disclosed. The external comprising an electrode configured to gather physiological data from a patient and a processor coupled to the electrode. The processor is configured to generate a sinusoidal waveform model of the physiological data, determine a feature from the model, and compare the feature to a standard to determine whether a shock is needed.

### BRIEF DESCRIPTION OF THE DRAWINGS

The present invention will hereinafter be described in conjunction with the following drawing figures, wherein like numerals denote like elements, and:

FIG. 1 illustrates an exemplary embodiment of a system for delivering a defibrillating pulse;

FIG. 2 is a simplified block diagram of an external defibrillator in accordance with an exemplary embodiment of the present invention;

FIG. 3 is a block diagram of the shock success prediction algorithm in accordance with an exemplary embodiment of the present invention;

FIG. 4 is a flowchart of a method for determining whether to provide an electric shock in accordance with an exemplary embodiment of the present invention; and

FIG. 5 is a flowchart of a method for determining whether to provide an electric shock based on the harmonic decomposition of a model of ventricular fibrillation in accordance with an exemplary embodiment of the present invention.

### DETAILED DESCRIPTION OF THE DRAWINGS

The following detailed description of the invention is merely exemplary in nature and is not intended to limit the invention or the application and uses of the invention. Furthermore, there is no intention to be bound by any expressed or implied theory presented in the preceding background of the invention or the following detailed description of the invention.

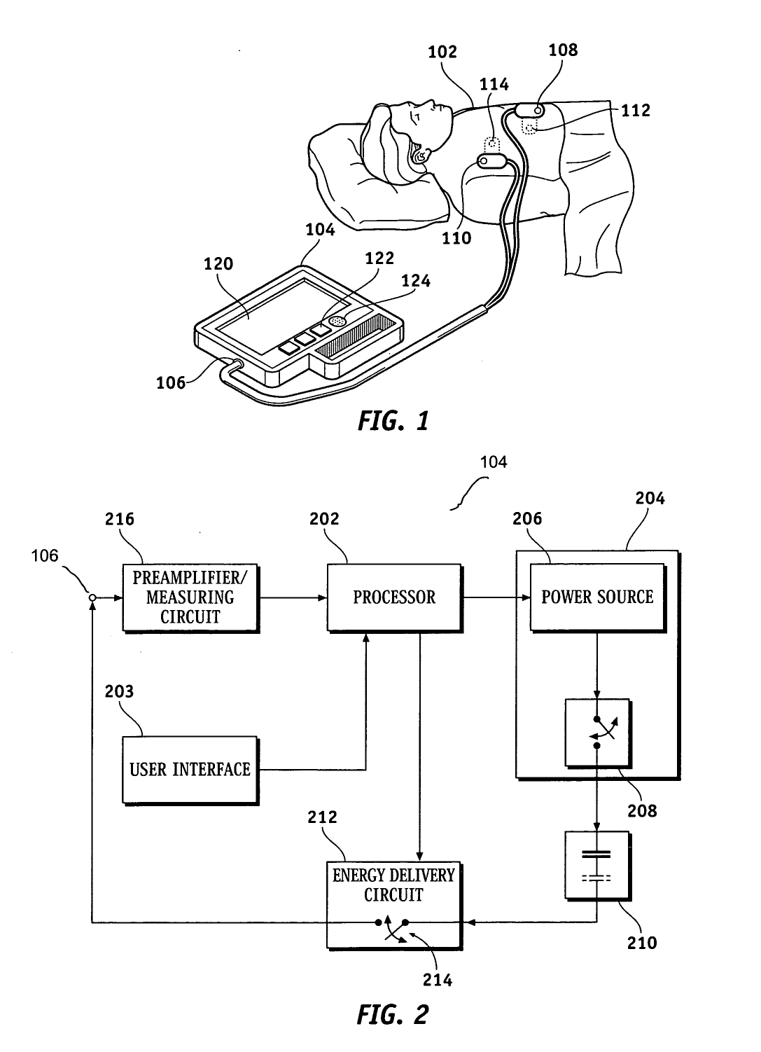

FIG. 1 illustrates an external defibrillator 104 that is configured to deliver a defibrillation shock to a patient 102, such as a patient undergoing ventricular fibrillation (VF). The external defibrillator 104 can be any number of external defibrillators in accordance with the present invention. For example, the external defibrillator 104 can be an Automatic External Defibrillator or Automated External Defibrillator (AED), semi-Automatic or semi-Automated External Defibrillator, or a manually operated external defibrillator. U.S. Pat. No. 4,610,254, which was issued to Morgan et al on Sep. 9, 1986, and U.S. Pat. No. 6,334,070, which was issued to Morgan et al on Dec. 25, 2001, provides illustrative examples of defibrillators, and these two patents are hereby incorporated in their entirety by reference. (As used herein, an automatic or automated activity occurs without human intervention.)

In an exemplary embodiment, the external defibrillator 104 preferably includes at least one connection port 106 for coupling one or more electrodes (108, 110) that are configured to deliver the defibrillation shock (also known as a defibrillation pulse) from the patient 102 to the external defibrillator 104. In addition, the one or more electrodes (108, 110), and/or other sensing electrodes (112, 114), are configured to sense physiological signals of the patient 102.

In an exemplary embodiment, the external defibrillator 104 can include a display 120 that is configured to visually present various measured or calculated parameters of patient 102 and/or other information to the operator (not shown) of the external defibrillator 104. For example, the display 120 can be configured to visually present the transthoracic impedance, electrocardiogram (ECG) and/or other physiology signals of the patient. The external defibrillator 104 can also include one or more input devices (e.g., switches or buttons) 122 that are configured to receive commands or information from the operator. A speaker 124 can also be included with the external defibrillator 104 to provide an audio output for instructions or other messages.

In an exemplary embodiment, the one or more electrodes (108, 110) and/or one or more sensing electrodes (112, 114) are configured to sense one or more physiological and/or physical parameters of the patient 102 that are received by the external defibrillator 104 at the connection port 106. Any number of physiological signals of the patient 102 can be sensed by the external defibrillator 104 with the one or more electrodes (108, 110) or the other sensing electrodes (112, 114). For example, conventional phonocardiogram (PCG) transducers can be used to convert acoustical energy of the patient's heart to electrical energy for production of a PCG waveform and/or the electrical activity of the patient's heart can be converted for production of an electrocardiogram (ECG) waveform. (See U.S. Pat. No. 5,687,738, which was issued to Shapiro et al on Nov. 18, 1997 and U.S. Pat. No. 4,548,204, which was issued to Groch et al on Oct. 22, 1985, for illustrative examples of detecting and displaying a PCG waveform, which are hereby incorporated in their entirety by reference. See also U.S. Pat. No. 4,610,254 as previously referenced and incorporated by reference for an illustrative example of obtaining and processing ECG data.) In a typical embodiment, the physiological data is comprised of data sampled at regular intervals for a set period of time. The PCG waveform, the ECG waveform, some other physiological signal or waveform of the patient 102, or a combination of more than one of these waveforms or signals is provided to defibrillator 104.

Referring to FIG. 2, a simplified block diagram of the external defibrillator 104 is illustrated in accordance with an exemplary embodiment of the present invention. The external defibrillator 104 preferably includes a processor 202, a user interface 203, which can comprise input devices 122 and display 120, a pre-amplifier/measuring circuit 216, a charging mechanism 204 that can include a power source 206 and a switch 208 to couple the power source 206 to the one or more energy storage devices (e.g., capacitors) 210 and an energy delivery circuit 212, which is illustrated as a switch 214 that is configured to selectively couple the one or more energy storage devices 210 to the connection port 106 under the control of the processor 202. The energy delivery circuit 212 can be implemented with any number of circuit configurations. For example, in a biphasic circuit, an H-bridge circuit can be used in accordance with the present invention.

The processor 202 preferably evaluates the one or more physiological signals of the patient 102 in accordance with executable instructions stored in a memory (not shown) of the external defibrillator 104 to determine, among other things, whether a defibrillation pulse (also referred to as a shock) should be applied to the patient 102, the parameters of the defibrillation pulse (e.g., pulse magnitude and duration), and the waveform parameters of the defibrillation shock (e.g., sinusoidal, monophasic, biphasic, truncated). The processor 202 can be a single processing unit or multiple processing units having one or more memories or the processor can be implemented as electronic circuitry, digital logic, software, or a combination of software/hardware configured to perform these activities and other activities of the external defibrillator 104.

The processor 202 can visually report the results or a portion of the signal detection results using a display 120. The display 120 can be any number of display configurations (e.g., Liquid Crystal Display (LCD) or Active Matrix Liquid Crystal Display (AMLCD) or can be a printer (not shown). Furthermore, the processor 202 can audibly report the results or a portion of the results to the operator using the speaker 124, which can be any number of audio generation devices. The processor 202 can also receive input from an operator (not shown) of the external defibrillator 104 via the user interface 203 which can include input devices 122 (e.g. keys, switches, buttons, or other types of user input).

In one embodiment, when the processor 202 determines that the application of a defibrillation pulse is beneficial for the patient 102, the energy storage device 210 (e.g. the defibrillation capacitors) of the external defibrillator 104 are charged, in one embodiment, by coupling the power source 206 of the charging mechanism 204 to the energy storage devices 210 via the switch 210. When the energy storage device 210m is charged, the processor 202 can visually or audibly advises the operator that the external defibrillator 104 is ready to deliver the defibrillation pulse. In one embodiment, the processor 202 requests operator initiation of the defibrillation pulse. When the operator requests the delivery of the defibrillation pulse, by, for example, pressing the input device 122 of the user interface 203, the processor 202 initiates a discharge of the energy stored in the energy storage device 210 by coupling the energy storage device 210 to the connection port 106 via the energy delivery circuit 210. The pulse is delivered to the patient via the electrodes 108, 110. In an alternative embodiment, the processor 202 can initiate the delivery of the defibrillation pulse without operator interaction when specified conditions are met (e.g., expiration of a predetermined period of time, acceptable measured patient impedance, etc.).

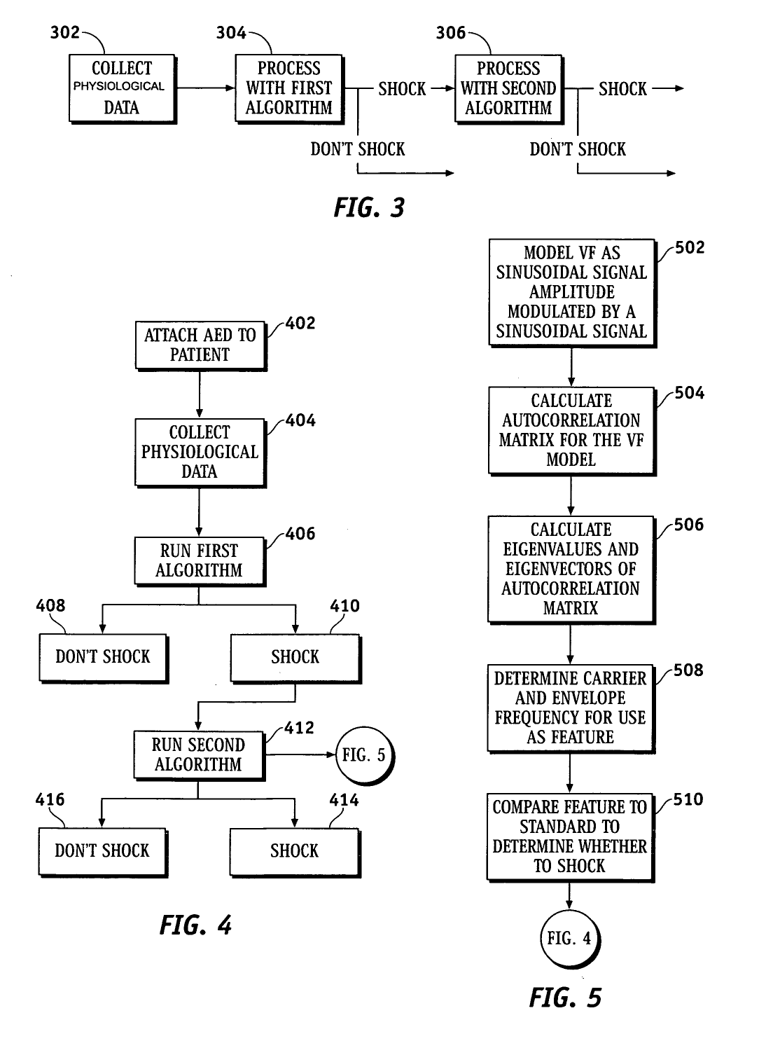

A method to determine if a shock should be initiated from the external defibrillator 104 is discussed with reference to FIGS. 3-5. In FIG. 3, physiological data 302 collected from the patient 102 is analyzed by a first algorithm 304. First algorithm 304 can be one of a number of known algorithms, such as those that implement fast Fourier transforms to evaluate ECG readings collected by the external defibrillator 104. As discussed previously, the physiological data 302 comprises data sampled at a certain sampling rate for a period of time. The output of first algorithm 304 is either a shock (and any necessary shock parameters) or a no-shock decision. In prior art defibrillators, the process ends here. However, in one embodiment of the present invention, a second algorithm 306 is used to review the shock decision of the first algorithm 304.

In one embodiment of the present invention, the second algorithm 306 determines the frequency associated with the VF waveform and compares them to a known standard to determine whether to shock. In this embodiment, the second algorithm models a VF waveform as a sinusoidal function and analyzes the frequency response of that function using a harmonic decomposition of the signal model. In an embodiment of the present invention, it is noted that the VF signal as seen on an ECG resembles a sinusoidal shaped signal that is amplitude modulated by a lower frequency sinusoidal signal. Thus, the VF signal can be modeled as a signal having a carrier frequency, f_{c}, and an envelope frequency, fₑ:
x(n)=Asin(2πf_{c}n+θ₁)sin(2πfₑn+θ₂)+w(n) (1)

The initial phases, θ₁ and θ₂, are independent, uniform random variables, w(n) is noise, f_{c} is the carrier frequency and fₑ is the envelope frequency (f_{c}≧fₑ). x(n) is a random variable. Eqn. 1 can be written as a sum of sine waves:

$$x ( n ) = \frac{A}{2} sin ( 2 π {f}_{1} n + {θ}_{3} ) + \frac{A}{2} sin ( 2 π {f}_{2} n + {θ}_{4} ) + w ( n ) \qquad ( 2 )$$

where f₁=f_{c}+fₑ and f₂=f_{c}−fₑ.

In order to determine the frequencies f₁ and f₂, the signal model of Eqn. 1 can be evaluated using the well known methods of harmonic decomposition. In the method, the sinusoidal signal model of Eqn. 1 can be represented as a complex sinusoidal signal model:

$$x ( n ) = {\sum }_{i = 1}^{P} {A}_{i} exp ( 2 jπ {f}_{i} n + {θ}_{i} ) + w ( n ) \qquad ( 3 )$$

The x(n)s are the individual data points that comprises the physiological data 302 as sampled from the individual. Using an exponential representation of the sinusoidal signal helps to simplify the calculations. The relationship between the random variables, x(n), can be examined using the autocorrelation function of x(n). The autocorrelation function is the expected value of the product of a random variable or signal with a time-shifted version of itself. The autocorrelation function can reveal if a process has a periodic component and the expected frequency of the periodic process. The expected value of x(n) can be expressed as:
ε(x(n+k)x*(n)))=r(k)=ΣAᵢ² exp(2jπfᵢk)+σ²δ(k) (4)
where ε is the expectation operation and σ² is the variance of the noise. The correlation function can be represented in matrix notation. Using an M×M autocorrelation matrix:

$$R = [ \begin{matrix}r ( 0 ) & r ( 1 ) & ⋯ & r ( M - 1 ) \\ r ( 1 ) & r ( 0 ) & ⋯ & r ( M - 2 ) \\ ⋮ & ⋮ & ⋯ & ⋮ \\ r ( M - 1 ) & r ( M - 2 ) & ⋯ & r ( 0 )\end{matrix} ] = {\sum }_{i = 1}^{P} {A}_{i}^{2} {s}_{i} {s}_{i}^{* T} + {σ}^{2} I \qquad ( 5 )$$

In the above matrix, sᵢ=[1e^{j2πfi}e^{2j2πfi} . . . e^{j2πPfi}]^{T} and I is the identity matrix. P is the vector space of the sine waves modeled in Eqn. 2 and 3. As seen in Eqn. 2, the VF is modeled as the sum of two real sine waves with frequencies f_{c} and fₑ. In this example P=4. P+1 to M represents the noise vector space. The eigenvectors of R can be denoted by uᵢ. For i=1, 2, . . . , P, the eigenvectors uᵢ span the vector space spanned by

$${\sum }_{i = 1}^{P} {A}_{i = 1}^{2} {s}_{i} {s}_{i}^{* T} .$$

For i=P+1, P+2, . . . , M, the eigenvectors, uᵢ, span the vector space spanned by σ²I (the noise space). The eigenvectors which span the sinusoidal waveform are orthogonal to the eigenvectors of the noise.

From the autocorrelation matrix and the above eigenvector calculations, an equation for frequency can be found. The frequencies can be estimated by:

$$\frac{1}{{\sum }_{i = P + 1}^{M} {\vert {r}^{* T} \cdot {u}_{i} \vert}^{2}} \qquad ( 6 )$$

where r=[1 e^{j2πf} e^{2j2πf} . . . e^{j2πMf}]^{T}. The frequency estimator of Eqn. 6 can be used to calculate a frequency for each of the signals described by Eqn. 2. The two dominant frequencies (the frequencies having the highest spectral peak energy) are chosen as the carrier frequency and the envelope frequency. The frequency in the expression for r is varied for a range of frequency, such as 0 to 25 Hz. The frequency range is chosen based on the expected frequencies seen in the VF. After calculating all of the frequencies, the highest peak frequency is chosen as the carrier frequency and the next highest as the envelope frequency. These frequencies can be used as features to determine whether to shock or not shock.

In order to determine whether to initiate a shock, the features can be compared to known standards. In one embodiment, the known standard is derived from analyzing multiple sets of data that are associated with either a case where it has been expertly determined that a shock should be applied or a case where it has been expertly determined not to shock. Each set of data is analyzed using the second algorithm and the carrier frequency, f_{c}, and the envelope frequency, fₑ, for each case is determined. The result is a collection of envelope frequencies, fₑ, and carrier frequencies, f_{c}, associated with either known shock or not shock cases. The collection forms a standard to which the carrier frequency, f_{c}, and envelope frequency, fₑ, determined from the data of a patient can be compared.

In one embodiment, the determined carrier frequency, f_{c}, and envelope frequency, fₑ, can be used as features to compare against the standard. In other embodiments, either the carrier frequency, f_{c}, or the envelope frequency, fₑ, can be used as the feature to compare against the standard. The comparison of the features to the standards can be done in one or many ways known in the art.

In another embodiment, the carrier frequency, f_{c}, and/or the envelope frequency, fₑ, can be used with other features to compare to the standard. One additional feature that can be used is the vector norm of the data, x(n). The vector norm is defined as:

$${‖ x ‖}_{p} = {( {\sum }_{n = 1}^{N} {\vert x ( n ) \vert}^{p} )}^{\frac{1}{p}} \qquad ( 7 )$$

While any vector norm can be calculated, using the L₁ norm and the L₂ norm is computationally simpler. The L₁ norm is defined as:

$${‖ x ‖}_{1} = {\sum }_{n = 1}^{N} \vert x ( n ) \vert \qquad ( 8 )$$

And the L₂ norm is defined as:

$${‖ x ‖}_{2} = \sqrt{{\sum }_{n = 1}^{N} {x ( n )}^{2}} \qquad ( 9 )$$

Either the L₁ or L₂ norm can be used in conjunction with the frequencies derived from the second algorithm. Of course, if the L₁ and/or L₂ norm is used as a feature to compare with the standard, the standard would have to have been derived using the L₁ and the L₂ norm along with any other feature being used.

FIG. 4 is a flowchart illustrating an exemplary method of determining whether to initiate a pulse in the external defibrillator 104. First, in step 402, the external defibrillator 104 is attached to the patient 102. Next, in step 404, physiological data is gathered from the individual. As discussed previously, the physiological data can be one or more parameters that can be used to determine if the individual's heart is in VF. The data is processed in step 406 using the first algorithm. As discussed previously, the first algorithm can be any known algorithm which can evaluate the physiological data and determine if a shock should be initiated. For example, the first algorithm could evaluate ECG readings from the heart using a Fourier transformation algorithm. If the result is not to shock (step 408), then the method ends and other resuscitation methods such as CPR can be used.

If a shock decision is made by the first algorithm (step 410), the physiological data is re-evaluated by the second algorithm in step 412. Referring to the flowchart of FIG. 5, an exemplary method for calculating the second algorithm is illustrated. In a first step, 502, the physiological data is modeled as a sinusoidal signal that is amplitude modulated by a lower frequency sinusoidal and characterized by a carrier frequency, f_{c}, and an envelope frequency, fₑ. As discussed previously, the amplitude modulated signal can be expressed as a sum of sine waves. (See Eqn. 2). Next, in step 504, the autocorrelation matrix is evaluated using Eqn. 5 and the physiological data collected in step 402.

After the autocorrelation matrix is evaluated in step 504, the eigenvalues and the corresponding eigenvectors of the autocorrelation matrix are determined in step 506. The eigenvectors are then used to calculate a series of frequency using Eqn. 6. The two dominant frequencies calculated are selected as the carrier frequency, f_{c}, and the envelope frequency, fₑ in step 508.

The carrier frequency, f_{c}, and the envelope frequency, fₑ, can be used, either singularly or together as features to be compared against a standard. This comparison occurs at step 510. The result is either a decision to shock or a decision not to shock.

Turning back to FIG. 4, the result of the second algorithm is either to initiate a shock in step 414 or to not shock in step 416.

While the present invention has been discussed in the context of use after a first algorithm, the second algorithm can also be used as the only algorithm to evaluate data. While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed:

1. A method of operating an external defibrillator configured to provide a defibrillation shock to a patient comprising:
gathering physiological data;
analyzing the physiological data using a first algorithm to determine whether to initiate a shock; and
if it is determined that the defibrillation shock should be provided, analyzing the physiological data using a second algorithm to verify the determination to shock.

2. The method of claim 1 wherein the step of gathering physiological data further comprises gathering physiological data concerning an electrical activity of the heart.

3. The method of claim 1, wherein the step of analyzing the physical data using a second algorithm further comprises forming a ventricular fibrillation model using the physiological data.

4. The method of claim 3, wherein the step of forming a ventricular fibrillation model further comprises modeling ventricular fibrillation as a sinusoidal signal that is amplitude modulated by a second sinusoidal signal.

5. The method of claim 4, wherein the step of analyzing the physical data using a second algorithm further comprises using a harmonic decomposition of the ventricular fibrillation model.

6. The method of claim 1, wherein the step of analyzing the physical data using a second algorithm to verify the determination to shock further comprises deriving features from the physiological data for use in determining whether to shock.

7. The method of claim 6 wherein the step of deriving features from the physiological data for use in determining whether to shock further comprises extracting a carrier frequency and an envelope frequency a sinusoidal model of ventricular fibrillation.

8. An external defibrillator comprising:
an electrode configured to gather physiological data from a patient;
a processor coupled to the electrode, the processor configured to:
(i) generate a sinusoidal waveform model of the physiological data;
(ii) determine a feature from the model; and
(iii) compare the feature to a standard to determine whether a shock is needed.

9. The external defibrillator of claim 8 wherein the processor is further configured to calculate an autocorrelation matrix from the sinusoidal waveform model and the physiological data.

10. The external defibrillator of claim 9 wherein a carrier frequency and an envelope frequency can be calculated for the sinusoidal waveform model and physiological data using eigenvectors of the autocorrelation matrix and wherein the carrier frequency and the envelope frequency comprising the features.

11. The external defibrillator of claim 10 wherein an Lₙ-norm of the physiological data can be used as an additional feature to compare to a standard.

12. The external defibrillator of claim 8 wherein the processor is configured to execute a first algorithm to determine whether to shock prior to generating the model of the physiological data.

13. The external defibrillator of claim 8 wherein the physiological data comprises a measurement of an electrical activity of the heart.

14. A method for determining whether to initiate a shock in a defibrillator having sensor paddles attached to a patient, the method comprising:
gathering physiological data regarding the patient;
modeling the physiological data using a sinusoidal waveform model;
determining a feature from the model; and
comparing the feature to a standard to determine whether a shock is needed.

15. The method of claim 14 wherein the step of gathering physiological data further comprises gathering physiological data concerning an electrical activity of the heart.

16. The method of claim 14, wherein the step of modeling the physiological data using a sinusoidal waveform model comprises modeling the physiological data as a sinusoidal shaped waveform amplitude modulated by a second sinusoidal signal.

17. The method of claim 14 wherein the step determining a feature from the model further comprises using a harmonic decomposition of the sinusoidal waveform model.

18. The method of claim 14, wherein the step of determining a feature from the model comprises deriving features from the physiological data for use in determining whether to shock.

19. The method of claim 18 wherein the step of deriving features from the physiological data for use in determining whether to shock further comprises extracting a carrier frequency and an envelope frequency for the sinusoidal waveform model for use as the features.

20. The method of claim 14 further comprising the step of using a first algorithm to determine if a shock should be initiated prior to the step of modeling the physiological data using a sinusoidal waveform model.
